(19) United States
(12) Patent Application Publication
(10) Pub. No.: US 2026/0114712 A1
(43) Pub. Date: Apr. 30, 2026

(54) TUBING ARRANGEMENT FOR MEDICAL APPLICATIONS AND METHOD FOR ITS PRODUCTION

(71) Applicant: Witzenmann GmbH, Pforzheim (DE)

(72) Inventors: Michael BREISINGER, Karlsbad (DE); Julius HEEL, Pforzheim (DE)

(73) Assignee: Witzenmann GmbH, Pforzheim (DE)

(21) Appl. No.: 19/371,057

(22) Filed: Oct. 28, 2025

(30) Foreign Application Priority Data
Oct. 28, 2024 (DE) 102024131393.5

(51) Int. Cl. A61B 1/00; B24C 1/06

(52) U.S. Cl. CPC A61B 1/00078; A61B 1/0011; A61B 1/00128; B24C 1/06

(57) ABSTRACT

A tube arrangement for medical applications, such as an endoscopy protective tube, has at least one metal coupling part, a metal wound tube, a braided tube and a kink protection sleeve. The wound tube is connected to the coupling part and the coupling part has a hole in communication with an interior of the wound tube. The braided tube surrounds the wound tube at least in one portion. The coupling part has an axial extension, with which it engages axially and at a radial distance over at least the wound tube and preferably also over the braided tube. the kink protection sleeve externally encloses the wound tube and preferably the braided tube fully circumferentially and extends both radially inside and radially outside the axial extension in the region of the coupling part. A production method for a tube arrangement is also provided.

### CROSS-REFERENCE TO RELATED APPLICATIONS

This application claims priority from German Patent Application No. DE 102024131 393.5, filed October28, 2024, which is incorporated herein by reference as if fully set forth.

### TECHNICAL FIELD

The invention relates to a tube arrangement for medical applications, in particular an endoscopy protective tube, having at least one metal coupling part, a metal wound tube, a braided tube and a kink protection sleeve.

The invention also relates to a method for producing the tube arrangement according to the invention.

### BACKGROUND

Such tube arrangements are known. They are used in particular to protect optical conductors (fiber-optic cables) and optionally further conduction elements, which are guided through the coupling part and the wound tube and are correspondingly protected against damaging effects from the outside. The coupling part may be used to connect the tube arrangement to a medical device, for example an endoscopy device. In previously known tube arrangements of the type mentioned, the coupling part has an axial extension with which it engages axially over the wound tube and preferably – if there is one – also over a braided tube. This axial extension is regularly configured in the form of a smooth cylindrical sleeve.

In previously known tube arrangements of the type mentioned, the aforementioned kink protection regularly consists of a silicone coating (so-called tube extrudate), which surrounds the wound tube and onto which an additional separate molded part (likewise regularly consisting of silicone) is molded in the region of the coupling part, where the risk of kinking is greatest, and is held externally on the coupling part with a form fit or force fit, there being a gap between the two silicone parts.

A problem with previously known tube arrangements of the type mentioned is that there are stringent hygiene requirements especially in medical technology, and therefore correspondingly stringent requirements for the leak-tightness of the aforementioned gap between the tube extrudate and the molded part. This gap, however, is in any case a potential or demonstrable site for deposits of moisture, dirt, particles or in general contaminants etc., which may accumulate there and form dangerous bacterial growths, all the more so because cleaning the aforementioned location is impossible or difficult, and correspondingly time-consuming.

### SUMMARY

The object of the invention is to alleviate this problem and to provide a tube arrangement which is less susceptible to the accumulation of contaminants and can be cleansed more easily, but nevertheless offers sufficient kink protection.

This object is achieved according to the invention by a tube arrangement having one or more of the features disclosed herein and by a related production method having one or more of the features disclose herein related to the method. Advantageous embodiments are defined in the following description and claims.

A tube arrangement according to the invention for medical applications, in particular an endoscopy protective tube, comprises at least one metal coupling part, a metal wound tube, a braided tube and a kink protection sleeve. The wound tube and the coupling part preferentially consist of (stainless) steel, while the braided tube may consist of glass silk. The wound tube is connected to the coupling part and the coupling part has a hole, which is in communication with an interior of the wound tube. The braided tube surrounds the wound tube at least in one portion, preferably over its entire length. The coupling part has an axial extension, with which it engages axially and at a radial distance over at least the wound tube and preferably also – if there is one – over the braided tube. The kink protection sleeve externally encloses the wound tube and preferably also – if there is one – the braided tube fully circumferentially and extends both radially inside and radially outside the axial extension in the region of the coupling part.

The terms “axial” and “radial” refer to a longitudinal axis of the tube arrangement when it has a straight profile, or is correspondingly arranged, “axial” denoting a direction along or parallel to the longitudinal axis and “radial” denoting a direction perpendicular thereto.

In a method according to the invention for producing the tube arrangement,

a) the wound tube is optionally surrounded with the braided tube;

b) the wound tube (and the braided tube, if there is one) is provided with a first kink protection sleeve;

c) the coupling part is placed onto the wound tube (and the braided tube, if there is one) and the first kink protection sleeve in the region of its axial extension;

d) the coupling part in the region of its axial extension and the first kink protection sleeve at least in a portion adjacent to the axial extension are externally provided with a second kink protection sleeve, which is materially connected to the first kink protection sleeve in order to form the kink protection sleeve.

The invention therefore comprises, or establishes, a full material connection between the materials (for example silicones) of the first and second kink protection sleeves, i.e. between the tube extrudate and the molded part, so that a one-piece, integral kink protection sleeve that no longer has a gap is obtained. Bacterial growths are thereby avoided and cleaning is simplified, without reductions in the degree of kink protection that can be achieved.

Preferentially, by using suitable formulations and the production process according to the invention, full crosslinking of the silicone materials (or comparable polymers) of the first and second kink protection sleeves during production takes place only after the application of the second kink protection sleeve, which ensures that the gap between the extrudate (first kink protection sleeve) and the molded part (second kink protection sleeve) is closed.

In one embodiment of the invention, the durability of the tube arrangement may be improved significantly by a preferred revision of the coupling part in relation to its previously known design. Especially, so-called edges or projections may be provided on the coupling part (in the region of the axial extension) as purchase for the polymer material (in particular silicone) during the molding process. Further, the coupling part may have an inner contour (in the region of the axial extension) as an incision element for the mounting on the wound tube provided with the first kink protection sleeve.

It is particularly advantageous to form a material connection between the molded part (second kink protection sleeve), which is preferentially present only in the region of the coupling part and a region directly adjacent axially, and the tube extrudate (first kink protection sleeve), which is preferentially present continuously over the entire wound tube.

The embodiments described below have proven particularly advantageous in practice:

In one development of the tube arrangement according to the invention, the kink protection sleeve is formed from a chemically crosslinkable (polymer) material, preferably a silicone material.

Such materials, in particular medical silicone, have become established for applications in the field of medical technology.

Since the desired one-piece configuration of the kink protection sleeve may be achieved particularly well when using such materials, according to another development of the tube arrangement according to the invention the kink protection sleeve is configured in one piece, which may be achieved by corresponding crosslinking. Such crosslinking ensures that the kink protection sleeve, which extends both radially inside and radially outside the axial extension in the region of the coupling part, forms an integral unit together with the further-continuing sleeve of the wound tube, so that there is no longer a gap that is susceptible to contamination.

In yet another development of the tube arrangement according to the invention, the coupling part has at least one structuring of its outer surface on the axial extension.

This structuring ensures better purchase of the kink protection material in the relevant region, which improves the durability and strength of the tube arrangement.

In yet another development of the tube arrangement according to the invention, the structuring has at least one first circumferential radial projection, preferably a plurality of first circumferential radial projections spaced apart axially.

Such projections may be produced easily and with a flexible geometry by known machining or cutting production methods, which simplifies the production of the tube arrangement.

In a corresponding development of the method according to the invention, the outer structuring is produced by a machining method or by a cutting method.

In yet another development of the tube arrangement according to the invention, the first projection has a sawtooth shape in longitudinal section. A steeper edge of the sawtooth shape, which for example is oriented at an angle of 90 degrees with respect to the longitudinal axis, may in this case face toward a free end of the coupling part, i.e. the coupling end. At the other end of the projection, the corresponding angle may – without restriction – be for example 30 ± 2 degrees with respect to the longitudinal axis.

Applicant has discovered that a particularly good durability and strength of the tube arrangement can be achieved with such a design.

In one advantageous development of the tube arrangement according to the invention, in at least one region on the axial extension, the coupling part externally has a roughened surface, a corrugation or a knurling. This may preferably be arranged between two first projections.

In a corresponding development of the method according to the invention, the roughened surface is produced by sandblasting.

Applicant has discovered that a particularly good durability and strength of the tube arrangement can also be achieved with such a surface configuration.

In one particularly advantageous development of the tube arrangement according to the invention, the coupling part has at least one structuring of its inner surface on the axial extension.

With this structuring, the coupling part can interact mechanically with the wound tube, or with its sleeve. Applicant has discovered that the durability and strength of the tube arrangement can be further improved in this way.

In another very particularly advantageous development of the tube arrangement according to the invention, the structuring (of the inner surface) has at least one second circumferential radial projection. There may preferably also be a plurality of second circumferential radial projections spaced apart axially. The projection, or the projections, is/are formed helically in the manner of at least one screw thread.

With this structuring, the coupling part may so to speak be screwed onto the wound tube, or onto its sleeve. Applicant has discovered that the durability and strength of the tube arrangement can be improved even further in this way.

Advantageously, the second projection may in this case cut into the kink protection sleeve, which additionally further improves the durability and strength of the tube arrangement.

According to a corresponding development of the method according to the invention, in step c) the coupling part is screwed with its screw thread, or the second projection, onto the first kink protection sleeve, with the second projection cutting into the first kink protection sleeve.

According to one particularly advantageous development of the method according to the invention, in step b) a chemically crosslinkable material, in particular a polymer material, preferably a silicone material, is applied, preferably extruded, as the first kink protection sleeve onto the wound tube. Furthermore, in step d) the first kink protection sleeve in the aforementioned portion, i.e. a portion adjacent to the axial extension, and the coupling part in the region of its axial extension are provided, preferably overmolded, with the second kink protection sleeve consisting of the same material or a compatible material. This is done before the material of the first kink protection sleeve is fully crosslinked, so that the two kink protection sleeves can be connected intimately to form a single kink protection sleeve.

Here, “compatible” means that the two materials can chemically crosslink in order to achieve the advantages associated therewith.

### BRIEF DESCRIPTION OF THE DRAWINGS

Further properties of the invention may be found in the following description of exemplary embodiments with the aid of the drawing. The same references denote elements that are identical or have the same effect.

FIG. 1 shows a perspective view of a tube arrangement according to the invention in longitudinal section; and

FIG. 2 shows the tube arrangement of FIG. 1 in longitudinal section and with two detail enlargements X, Y.

### DETAILED DESCRIPTION

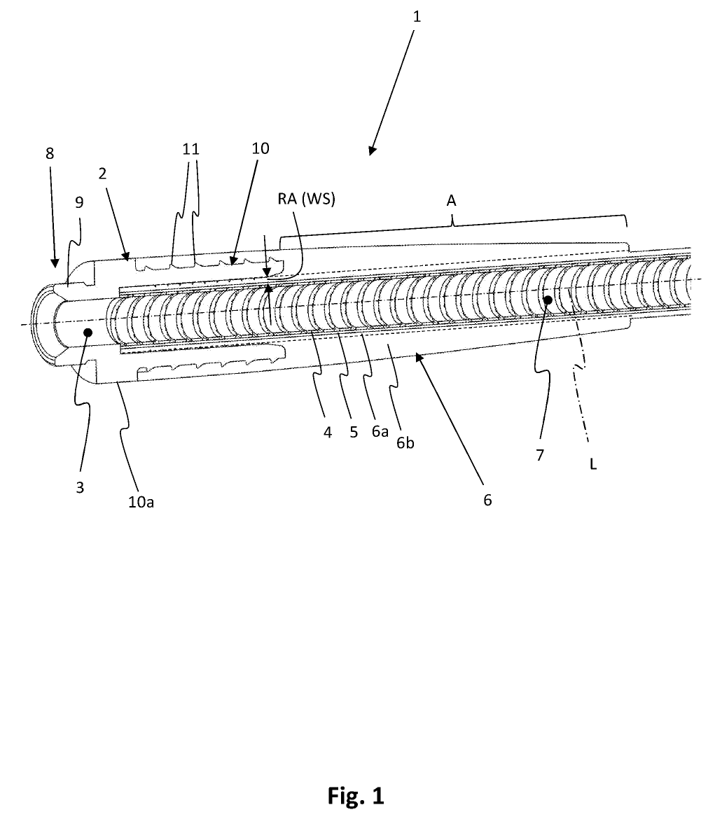

FIG. 1 shows a tube arrangement 1 according to the invention. Reference L denotes the longitudinal axis thereof. The tube arrangement 1 is suitable for medical applications and may, in particular, be used as an endoscopy protective tube. It comprises a metal coupling part 2, for example consisting of stainless steel, with a central hole 3. It further comprises a metal wound tube 4, preferably also consisting of stainless steel, which is enclosed by a braided tube 5 consisting of glass silk (a silklike material consisting of glass fibers). The braided tube 5 surrounds the wound tube 4 over its entire length. Furthermore, the tube arrangement 1 comprises a kink protection sleeve 6 consisting of crosslinked (medical) silicone.

As may be seen from FIG. 1, the wound tube 4 is connected to the coupling part 2, the coupling part 2 being in communication via its hole 3 with an interior 7 of the wound tube 4 in order, for example, to be able to insert fiber-optic cables (not shown) through the coupling part 2 into the wound tube 4.

At its free end 8, the coupling part 2 has an (outer) screw thread 9 by which it can be connected to a medical device, for instance an endoscopy device (not shown).

The coupling part 2 is preferably formed as a turned part, any swarf created during production having been removed by suitable cleaning, in particular internally in the region of the hole 3.

The coupling part 2 has an axial extension 10, with which it engages axially and at a radial distance RA, RA > 0, over the wound tube 4 and the braided tube 5. The kink protection sleeve 6 externally encloses the wound tube 4 and the braided tube 5 fully circumferentially and over the entire length of the tube arrangement 1 while extending both radially inside (i.e. between the wound tube 4, or the braided tube 5, and the axial extension 10) and radially outside the axial extension 10 in the region of the coupling part 2, and especially in the region of the axial extension 10 there. The kink protection sleeve 6 in this case forms a one-piece entity – without a gap or the like. A wall thickness WS of the kink protection sleeve 6 between the wound tube 4, or the braided tube 5, and the axial extension 10, is about 1 mm, which corresponds to a dimension of the aforementioned radial distance RA. Arranged axially between the axial extension 10 and the screw thread 9, there is a radial projection 10a.

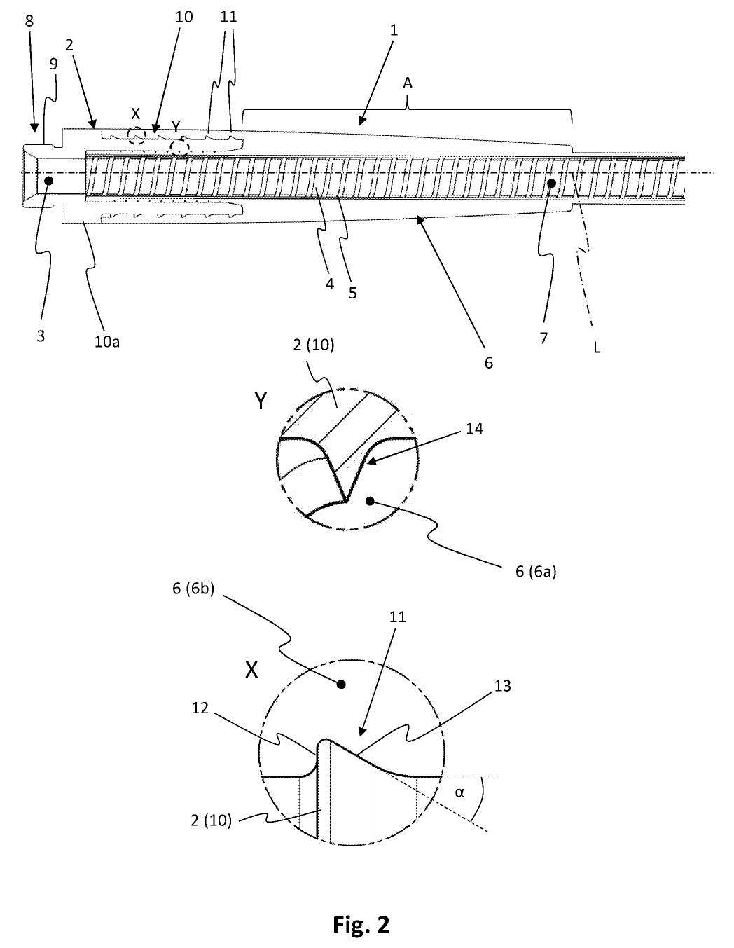

As may also be seen from FIG. 1, and as is explained in more detail below with the aid of FIG. 2, the coupling part 2 has at least one structuring of its outer surface on the axial extension 10. This structuring comprises a plurality of first circumferential radial projections spaced apart axially, some of which are denoted by the reference 11. The projections 11 have a sawtooth shape in longitudinal section, with the steeper edge of the sawtooth shape facing toward the free end 8 of the coupling part 2. This is visible particularly clearly in the detail excerpt X of FIG. 2, where the steeper edge of the sawtooth shape is provided with the reference 12 and extends perpendicularly with respect to the longitudinal axis L. The angle α between the longitudinal axis L and the other (shallower) edge 13 of the sawtooth shape is in the present case about 30 degrees.

In at least one region on the axial extension 10, the coupling part 2 may also externally have a roughened (for example by sandblasting) surface, a corrugation or a knurling, which cannot be seen in the figure. This additional structuring may preferably be arranged between two first projections 11.

In principle, an embodiment entirely without first projections 11 or with a reduced number of first projections 11 may also be envisioned here. FIG. 1 shows six first projections 11. According to another embodiment, for example, the four projections 11 in the middle may be eliminated and a knurling or a surface modified by sandblasting may instead be provided in this region.

As may moreover also be seen from FIG. 1, and as is explained in more detail below with the aid of FIG. 2, the coupling part 2 has at least one structuring of its inner surface on the axial extension 10. This structuring comprises a plurality of second circumferential radial projections 14 spaced apart axially, which are arranged helically in the manner of at least one screw thread on the inner side of the axial extension 10. This is visible particularly clearly in the detail excerpt Y of FIG. 2. The second projections 14 preferably protrude by about 0.2 to 0.6 mm radially inward in relation to the axial extension 10.

For example, they may be three spirally wound second projections 14, which each make three full circuits on the inner side of the axial extension 10 and which are arranged offset in the axial direction by one third of their respective (equal) pitch.

With the second projections 14, the coupling part 2 cuts into the kink protection sleeve 6. In this way, the tube arrangement 1 can withstand extraction forces of up to 150 N.

The following procedure may preferably be adopted during the production of the tube arrangement 1:

First, the wound tube 4 is surrounded in the desired way with the braided tube 5. The wound tube 4 together with the braided tube 5 is then provided with a first kink protection sleeve, which is part of the kink protection sleeve 6 already mentioned and in the present case also is/has been referred to as a tube extrudate because of the preferred type of production. In FIG. 1, this first kink protection sleeve is symbolically represented, or delimited, by means of a dashed line and denoted by the reference 6a. The coupling part 2 is next placed in the region of its axial extension 10 onto the wound tube 4 (with the braided tube 5) and the first kink protection sleeve 6a by screwing it with the second projections 14 onto the first kink protection sleeve 6a, with the aforementioned second projections 14 cutting into the first kink protection sleeve 6a. The coupling part 2 in the region of its axial extension 10 and the first kink protection sleeve 6a at least in a portion A adjacent to the axial extension 10 (cf. FIG. 2) are subsequently provided from the outside with a second kink protection sleeve 6b (cf. FIG. 1), which is materially connected to the first kink protection sleeve 6a in order to form a single one-piece kink protection sleeve 6 and extends as far as against the radial projection 10a. The first projections 11 externally on the axial extension 10 ensure a secure purchase of the kink protection material on the coupling part 2.

As has already been described, for this purpose a chemically crosslinkable material, in particular a polymer material, preferably a silicone material, is applied, preferably extruded, as the first kink protection sleeve 6a onto the wound tube 4 and the braided tube 5. Next, the first kink protection sleeve 6a in the aforementioned portion A and the coupling part 2 in the region of its axial extension 10 are provided with the second kink protection sleeve 6b, which consists of the same material as the first kink protection sleeve 6a or of a compatible material. This may be done by overmolding, and it takes place at a time before the material of the first kink protection sleeve 6a is fully crosslinked so that the integral kink protection sleeve 6 can be obtained from the first kink protection sleeve 6a and the second kink protection sleeve 6b.

At the other end (not shown), the tube arrangement 1 is preferentially formed in the same way as represented in FIGS. 1 and 2.

1. A tube arrangement (1) for medical applications, the tube arrangement (1) comprising:
at least one metal coupling part (2);
a metal wound tube (4);
a braided tube (5); and
a kink protection sleeve (6), wherein
the wound tube (4) is connected to the coupling part (2) and the coupling part (2) has a hole (3), which is in communication with an interior (7) of the wound tube (4);
the braided tube (5) surrounds the wound tube (4) at least in one portion;
the coupling part (2) has an axial extension (10), with which the coupling part (2) engages axially and at a radial distance (RA) over at least the wound tube (4); and
the kink protection sleeve (6) externally encloses the wound tube (4) fully circumferentially and extends both radially inside and radially outside the axial extension (10) in a region of the coupling part (2).

2. The tube arrangement (1) as claimed in claim 1, wherein the kink protection sleeve (6) is formed from a chemically crosslinkable material.

3. The tube arrangement (1) as claimed in claim 1, wherein the kink protection sleeve (6) is configured in one piece.

4. The tube arrangement (1) as claimed in claim 1, wherein the coupling part (2) has at least one structuring of an outer surface thereof on the axial extension (10).

5. The tube arrangement (1) as claimed in claim 4, wherein
the structuring has at least one first circumferential radial projection (11).

6. The tube arrangement (1) as claimed in claim 4, wherein the structuring has a plurality of first circumferential radial projections (11) spaced apart axially.

7. The tube arrangement (1) as claimed in claim 4, wherein
the first circumferential radial projection (11) has a sawtooth shape in longitudinal section, with a steeper edge (12) of the sawtooth shape facing toward a free end (8) of the coupling part (2).

8. The tube arrangement (1) as claimed in claim 4, wherein in at least one region on the axial extension (10), the coupling part (2) externally has a roughened surface, a corrugation or a knurling.

9. The tube arrangement (1) as claimed in claim 1, wherein the coupling part (2) has at least one structuring of an inner surface thereof on the axial extension (10).

10. The tube arrangement (1) as claimed in claim 9, wherein the at least one structuring has at least one second circumferential radial projection (14).

11. The tube arrangement (1) as claimed in claim 9, wherein the at least one structuring has a plurality of second circumferential radial projections (14) spaced apart axially, which are arranged helically as at least one screw thread.

12. The tube arrangement (1) as claimed in claim 11, wherein the second projection (14) cuts into the kink protection sleeve (6).

13. The tube arrangement (1) of claim 1, wherein the tube arrangement is an endoscopy protective tube.

14. The tube arrangement (1) of claim 1, wherein the axial extension (10) of the coupling part (2) engages axially and at the radial distance (RA) over at least the wound tube (4) and the braided tube (5).

15. The tube arrangement (1) of claim 1, wherein the kink protection sleeve (6) externally encloses the wound tube (4) and the braided tube (5) fully circumferentially.

16. A method for producing a tube arrangement (1), the method comprising:
a) at least partially surrounding a wound tube (4) with a braided tube (5);
b) providing the wound tube (4) with a first kink protection sleeve (6a);
c) placing a coupling part (2) onto the wound tube (4) and the first kink protection sleeve (6a) in a region of an axial extension (10) of the coupling part (2);
d) providing the coupling part (2) in the region of the axial extension (10) and the first kink protection sleeve (6a) at least in a portion (A) adjacent to the axial extension (10) externally with a second kink protection sleeve (6b), which is materially connected to the first kink protection sleeve (6a) in order to form a kink protection sleeve (6).

17. The method as claimed in claim 16, wherein
in step c) the coupling part (2) is screwed with a second projection (14) located on an inner surface of the axial extension (10) onto the first kink protection sleeve (6a), with the second projection (14) cutting into the first kink protection sleeve (6a).

18. The method as claimed in claim 16, further comprising machining an outer structuring on an outer surface of the coupling part (2) on the axial extension (10).

19. The method as claimed in claim 16, further comprising sandblasting an outer surface of the coupling part (2) on the axial extension (10) to form a roughened surface.

20. The method as claimed in claim 1, wherein
in step b) applying a chemically crosslinkable material onto the wound tube (4) as the first kink protection sleeve (6a); and
in step d) providing the first kink protection sleeve (6a) in the portion (A) adjacent to the axial extension (10) and the coupling part (2) in the region of the axial extension (10) with the second kink protection sleeve (6b) that is formed of a same material or a compatible material, before the chemically crosslinkable material of the first kink protection sleeve (6a) is fully crosslinked.
